(19) United States
(12) Patent Application Publication
(10) Pub. No.: US 2009/0326643 A1
(43) Pub. Date: Dec. 31, 2009

(54) BALLOON FOLDING APPARATUS AND METHOD

(75) Inventors: Rob Lucas, Princeton, MN (US); Mark Sagedahl, Maple Grove, MN (US); Adam Jennings, Buffalo, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/163,414

(22) Filed: Jun. 27, 2008

(51) Int. Cl. A61F 2/06; A61M 31/00

(52) U.S. Cl. 623/1.35; 623/1.11; 623/1.28; 623/1.12; 604/500

(57) ABSTRACT

A catheter assembly and related methods for preparing and assembling catheter assemblies. The catheter assembly includes main and side balloons. The main balloon includes side portions that are folded in opposite directions toward a bottom surface of the main balloon to place the main balloon in a folded state. The side balloon is typically positioned along a top surface of the main balloon. The folded balloons can be retained in a folded state with various retaining structures during further preparation and assembling of the catheter assembly.

### TECHNICAL FIELD

This disclosure relates to catheter assemblies configured for treatment of a vessel bifurcation, and more particularly relates to catheter balloon folding arrangements and related methods for such catheter assemblies.

### BACKGROUND

Catheters are used with stents and inflatable structures to treat conditions such as strictures, stenoses, and narrowing in various parts of the body. Various catheter designs have been developed for the dilatation of stenoses and to deliver and deploy stents at treatment sites within the body.

Stents are typically intraluminally placed by a catheter within a vein, artery, or other tubular shaped body organ for treating conditions such as, for example, occlusions, stenoses, aneurysms, dissections, or weakened, diseased, or abnormally dilated vessels or vessel walls, by expanding the vessels or by reinforcing the vessel walls. Once delivered, the stents can be expanded using one or more inflatable members such as balloons. Stents can improve angioplasty results by preventing elastic recoil and remodeling of the vessel wall and treating dissections in blood vessel walls caused by balloon angioplasty of coronary arteries. Stents can also be used as a drug delivery medium for treatment of damaged portions of a vessel.

While conventional stent technology is relatively well developed, stent technologies related to treatment of the region of a vessel bifurcation are still being developed. One challenge related to treatment of a vessel bifurcation involves protecting edges of the stent during delivery and repositioning of the stent to a vessel bifurcation treatment site.

### SUMMARY OF THE DISCLOSURE

The present disclosure relates to catheter assemblies and related methods of preparing and assembling catheter assemblies. One aspect of the present disclosure relates to folding arrangements for at least one balloon of a catheter assembly. Another aspect of the present disclosure relates to methods of folding a balloon and retaining the folded balloon during assembly of a plurality of components of the catheter assembly. Another aspect of the present disclosure relates to structures used to hold the balloon at various stages in the process of folding the balloon and assembling the catheter assembly.

The catheter assemblies, balloons, and related methods disclosed herein can be particularly suited for use in treating vessel bifurcations. In one example, the balloon is a main balloon of a catheter assembly, wherein the main balloon is configured to remain in a main vessel of a vessel bifurcation spanning across an opening or ostium into a branch vessel of the vessel bifurcation. The present disclosure can also relate to folding arrangements and related methods directed to a side balloon of the catheter assembly, wherein the side balloon is aligned with the ostium of the branch vessel and extends radially outward relative to the main balloon and through the ostium of the branch vessel.

There is no requirement that an arrangement include all features characterized herein to obtain some advantage according to this disclosure.

### BRIEF DESCRIPTION OF THE DRAWINGS

FIG. 1 is a schematic side view of an example balloon assembly in accordance with principles of the present disclosure with a main balloon and a side balloon inflated.

FIG. 2 is a schematic cross-sectional view taken along cross-sectional indicators 2-2 of FIG. 1.

FIG. 3 is a schematic side view of the balloon assembly shown in FIG. 1 with the main balloon in a compressed state and the side balloon deflated.

FIG. 4 is a schematic top view of the balloon assembly shown in FIG. 3.

FIG. 5 is a schematic cross-sectional view taken along cross-sectional indicators 5-5 of FIG. 4.

FIG. 6 is a schematic side view of the balloon assembly shown in FIG. 3 with side portions of the main balloon folded under.

FIG. 7 is a schematic cross-sectional view of the balloon assembly shown in FIG. 6 taken along cross-sectional indicators 7-7.

FIG. 8 is a schematic side view of the balloon assembly shown in FIG. 6 with the side balloon re-inflated and the main balloon having been further constricted to provide a reduced outer profile.

FIG. 9 is a schematic cross-sectional view of the balloon assembly shown in FIG. 8 taken along cross-sectional indicators 9-9.

FIG. 10 is a schematic side view of the balloon assembly shown in FIG. 8 with the side balloon in an alternative compressed state.

FIGS. 11A-11D are schematic top views of different folding arrangements for the side balloon.

FIG. 12 is a schematic side view of a catheter assembly that includes the balloon assembly shown in FIG. 1.

FIG. 13 is a schematic cross-sectional view of the catheter assembly shown in FIG. 12 taken along cross-sectional indicators 13-13.

FIG. 14 is a schematic cross-sectional view of the catheter assembly shown in FIG. 12 taken along cross-sectional indicators 14-14.

### DETAILED DESCRIPTION

This disclosure relates to bifurcation treatment systems, catheter assemblies, and related methods of treating bifurcations in a patient's body. The term bifurcation means a division location from one unit into two or more units. Generally, two types of bifurcations of a body organ include: 1) a main tubular member defining a main lumen and a branch tubular member defining a branch lumen that extends or branches off from the main tubular member, wherein the main and branch lumens are in fluid communication with each other, and 2) a primary or main member defining a primary or main lumen (also referred to as a parent lumen) that splits into first and second branch members defining first and second branch lumens. The term lumen means the cavity or bore of a tubular structure such as a tubular organ (e.g., a blood vessel).

An example bifurcation is a vessel bifurcation that includes a continuous main vessel and a branch vessel, wherein the vessels define a main lumen and a branch lumen, respectively that are in fluid communication with each other. Alternatively, a vessel bifurcation can include a parent vessel that divides into first and second branch vessels, wherein the vessels define a parent lumen and first and second branch lumens, respectively, which lumens are all in fluid communication with each other.

Example applications of the inventive principles disclosed herein include cardiac, coronary, renal, peripheral vascular, gastrointestinal, pulmonary, urinary, and neurovascular systems. The catheter assemblies, systems and methods disclosed herein can be used for locating a branch vessel of the vessel bifurcation and for placement of a stent relative to the vessel bifurcation for treatment of the vessel bifurcation.

Referring now to FIGS. 12-14, an example catheter assembly 10 is shown and described. The catheter assembly 10 includes a main catheter branch 12, a side catheter branch 14, a stent 16, a main guidewire 18, and a branch guidewire 20. The main catheter branch 12 includes a catheter shaft 22, a main guidewire housing 24 defining a main guidewire lumen 25, a main balloon 26, and a side balloon 28. The main and side balloons 26, 28 are shown in a compressed state in FIGS. 12-14.

The stent 16 includes proximal and distal end portions 80, 82, and a side branch aperture 84 positioned at a location between the proximal and distal end portions 80, 82. The main catheter branch 12 extends through an interior of the stent 16 from at least the proximal end portion 80 to the distal end portion 82. The side catheter branch 14 extends through a portion of the interior of the stent 16 from at least the proximal end portion 80 and out of the side branch aperture 84. The side catheter branch 14 defines a branch guidewire lumen 61 (see FIG. 12).

The catheter assembly 10 is adapted for treatment of a vessel bifurcation wherein the main guidewire 18 is first positioned in a main vessel of the vessel bifurcation extending to a location distal of an opening or ostium into the branch vessel, and the branch guidewire 20 extends through the ostium of the branch vessel and distally into the branch vessel. The assembly of the main catheter branch 12, side catheter branch 14, and stent 16 is then advanced over the guidewires 18, 20 to a location adjacent to the vessel bifurcation with the side balloon 28 positioned in axial and radial alignment with the ostium of the branch vessel. A distal end portion 60 of the side catheter branch 14 is positioned within the branch vessel. Positioning of the distal end portion 60 within the branch vessel helps to align the side balloon 28 with the ostium of the branch vessel and maintain such alignment during inflation of the main and side balloons 26, 28. Inflation of the main balloon 26 expands the stent 16 into engagement with the main vessel wall. The stent 16 can include expandable portions surrounding or defining the side branch aperture 84. This expandable portion can extend radially outward relative to the stent 16 upon inflation of the side balloon 28. Such expandable portions (not shown) can extend into the branch vessel to provide treatment in the area of the ostium of the branch vessel.

Maintaining alignment of the side balloon 28 with the ostium of the branch vessel during inflation of at least the main balloon 26 can be important for proper treatment of the vessel bifurcation. Some folding arrangements for the main balloon 26 can result in rotation of the main balloon during inflation, which in turn can result in radial movement of the side balloon 28 relative to the ostium of the branch vessel. The folding arrangement for the side balloon 28 can be influential in providing desired movement of the expandable portion of the stent 16 that defines the side branch aperture 84 properly into engagement with the ostium and other portions of a branch vessel of a vessel bifurcation being treated.

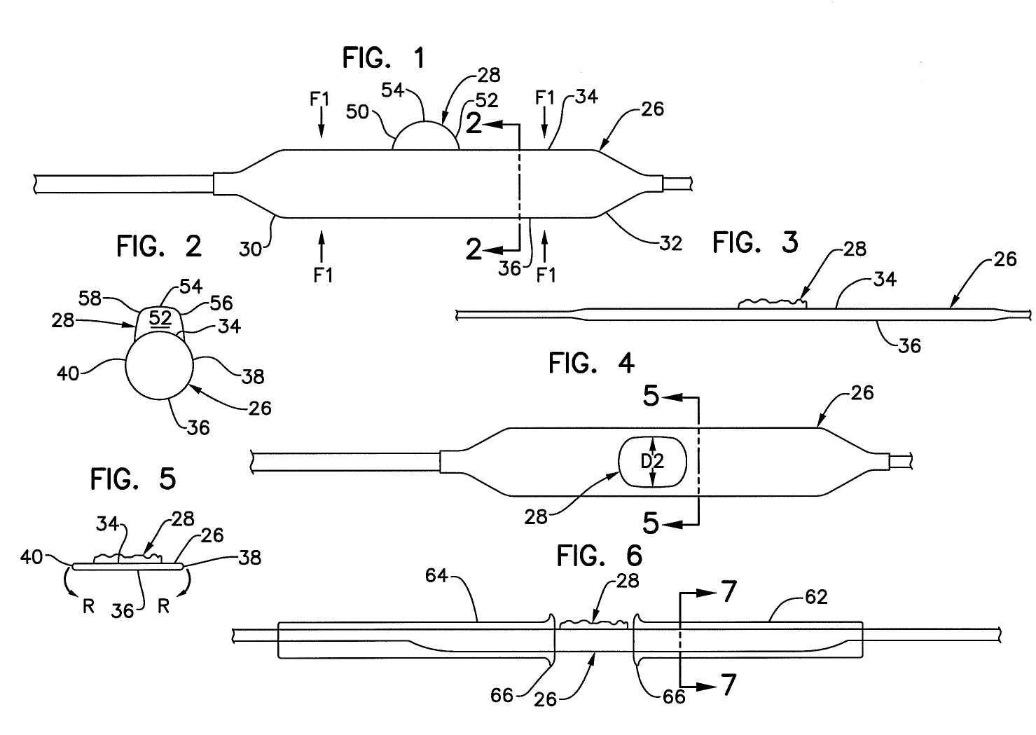

Referring now to FIGS. 1-11, some example folding arrangements and related methods of folding and assembling balloon members of a catheter assembly are described in further detail. FIGS. 1-5 illustrate an example balloon assembly that includes a main balloon 26 and a side balloon 28 in an inflated state. The main balloon 26 includes a proximal end portion 30, a distal end portion 32, a top side portion 34, a bottom side portion 36, and first and second side portions 38, 40.

The side balloon 28 is positioned along the top side portion 34 of the main balloon 26 at a location spaced between the proximal and distal end portions 30, 32. The side balloon 28 includes a proximal portion 50, a distal portion 52, a top portion 54, and first and second side portions 56, 58. The side balloon 28 can be integrally formed with the main balloon 26. In one example, the side balloon 28 is formed by a molding process directly from a portion of the main balloon 26. In other arrangements, the side balloon 28 can be formed as a separate piece and later attached to the main balloon 26 using any desired attachment techniques, such as, for example, laser welding or adhesives.

Referring again to FIG. 1, a method of folding a catheter balloon (e.g., main balloon 26) to provide a desired balloon folding arrangement is initiated by applying a compression force F1 to the top and bottom side portions 34, 36 to flatten the main balloon 26. In some cases, the force F1 can be applied only to the top side portion 34 while the bottom side portion 36 remains in engagement with a flat supporting surface. In other arrangements, the force F1 is applied to the bottom side portion 36 only while the top side portion 34 is maintained in engagement with a flat supporting surface. The force F1 is applied in a direction generally parallel to the direction in which the side balloon 28 extends radially outward from the top side portion 34 of the main balloon 26.

A vacuum force can be applied internally within the main and side balloons 26, 28 concurrently with application of the force F1 to help remove all fluids from within the main and side balloons 26, 28. Application of the vacuum force internal of the balloons 26, 28 during at least a portion of the time during which the force F1 is applied can help maximize flattening of the main balloon 26.

Application of the vacuum force internal of the main and side balloons 26, 28 tends to flatten and reduce the profile of the side balloon 28 without application of any external force to the side balloon 28. FIGS. 3-5 illustrate the main and side balloons in the compressed and/or flattened state. The vacuum force can be maintained for any desired time period, such as during subsequent steps of the balloon folding process, to minimize incidence of unintentional re-inflation of the main and side balloons 26, 28.

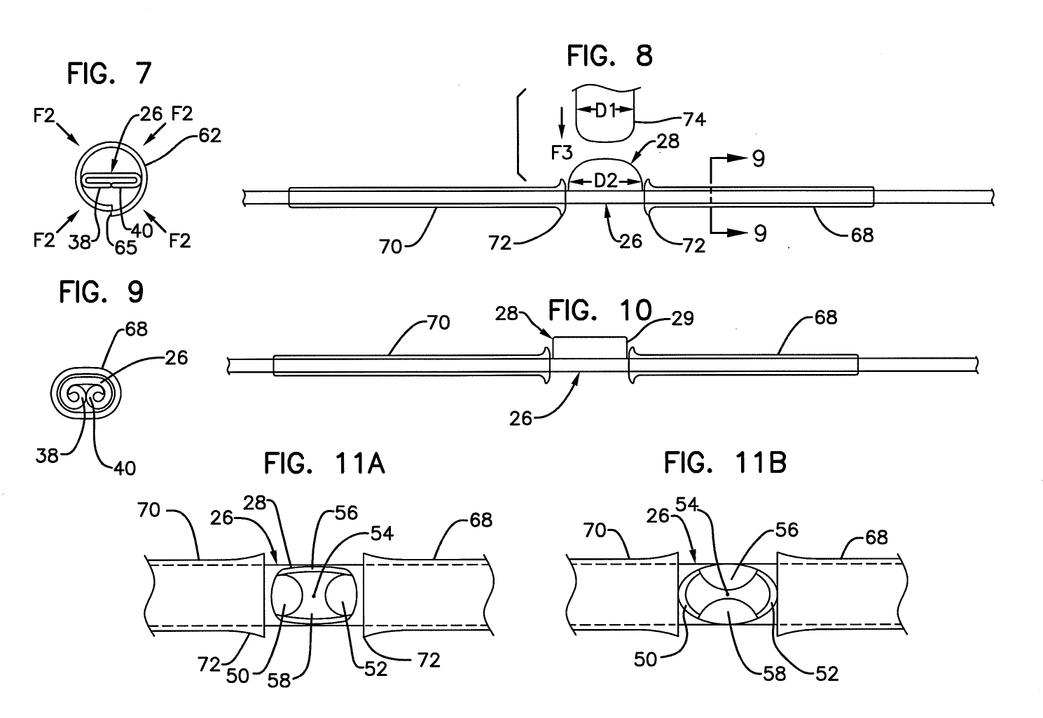

After flattening of the main balloon, the first and second side portions 38, 40 of the main balloon 26 are folded under in the radial direction R towards the bottom side portion 36 (see FIG. 5) to place the main balloon 26 in a first folded state shown in FIG. 7. With the main balloon 26 in this first folded state, a pair of first and second compressible holding members 62, 64 are advanced over the main balloon 26 from opposing sides of the side balloon 28. The compressible holding members 62, 64 each include a flared end portion 66 and a longitudinally arranged split 65 (see FIG. 7). The flared end portion 66 can help in advancing the compressible holding members 62, 64 over the main balloon 26. The flared end portion 66 can also provide an interface with the side balloon 28 that is less susceptible to damaging the side balloon 28 when, for example, the side balloon 28 is inflated as shown in FIG. 8.

The split 65 permits radially inward compression of the compressible holding members 62, 64 upon application of a radially inward directed force F2 (see FIG. 7). Applying the force F2 further compresses the main balloon 26 to provide a reduced outer profile of the main balloon 26. FIG. 9 illustrates the main balloon 26 after having been compressed by application of the force F2 to provide a reduced outer profile. FIG. 9 illustrates the side portions 38, 40 in a further rolled under configuration. In some arrangements, the side portions 38, 40 move into different configurations when the force F2 is applied to the compressible holding members 62, 64.

With the main and side balloons 26, 28 in this reduced outer profile state, the first and second compressible holding members 62, 64 are removed and a pair of first and second non-compressible holding members 68, 70 are advanced over the main balloon 26 as shown in FIG. 8. The non-compressible holding members 68, 70 can also include flared end portion 72. The non-compressible holding members 68, 70 have a shape and size that helps maintain the main balloon 26 in the reduced profile state (see FIG. 9) during further folding steps of the side balloon 28 and assembly of the balloons 26, 28 with other components of the catheter assembly 10.

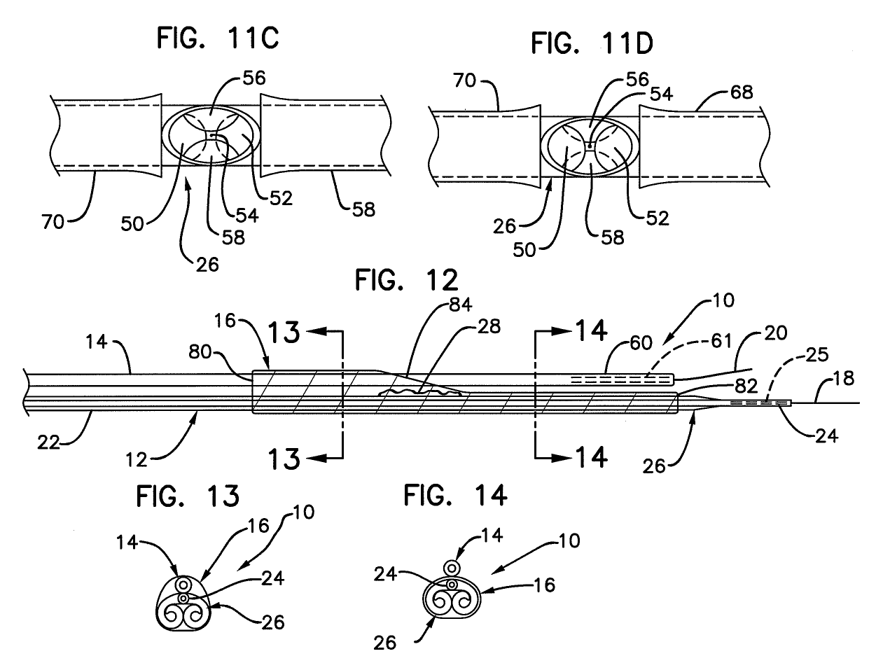

With the main balloon 26 in the compressed reduced profile state held by the non-compressible holding members 68, 70, the side balloon 28 can be re-inflated as an initial step in providing a specific folding configuration for the side balloon 28. With the side balloon 28 inflated as shown in FIG. 8, a force F3 applied by a compressible member 74. The compressible member 74 can have an outer profile dimension D1 that is less than an outer profile dimension D2 of the side balloon 28 is applied to the top portion 52 of the side balloon 28. The compressible member 74 creates a crater-like configuration for the side balloon 28 wherein the side balloon 28 has a lip structure arranged around its periphery. This lip structure can be folded into different folded configurations as shown with reference to FIGS. 11A-11D.

In other arrangements, the compressible member 74 can have dimensions D1 that are greater than dimensions D2 to essentially flatten the entire side balloon 28 in a non-specific folded arrangement against the main balloon 26. In still further arrangements, the compressible member 74 can have a dimension that is greater in a transverse direction than in a longitudinal direction relative to the longitudinal axis of the main balloon 26. This type of construction for the compressible member 74 provides flattening the first and second side surfaces 56, 58 of the side balloon 28 against the main balloon 28 while the proximal and distal portions 50, 52 remain in a raised arrangement that can be folded downward onto the main balloon 26. Various other configurations, shapes and sizes for the compressible member 74 can be used for flattening and folding portions of the side balloon 28.

FIG. 11A illustrates the proximal and distal portions 50, 52 of the side balloon 28 folded downward onto the main balloon 26 while the first and second side portions 56, 58 remain either flattened or partially raised relative to the top side 34 of the main balloon 26. FIG. 11B illustrates the first and second side portions 56, 58 folded onto the main balloon 28 while the proximal and distal portions 50, 52 remain either flattened or partially raised relative to the top side 34 of the main balloon 26. FIG. 11C illustrates the proximal and distal portions 50, 52 folded first upon the main balloon 26 followed by folding of the first and second side portions 56, 58 onto the proximal and distal portions 50, 52. FIG. 11 illustrates the first and second side portions 56, 58 first folded down onto the main balloon 26 followed by folding of the proximal and distal portions 50, 52 onto the first and second side portions 56, 58.

After the main balloon 26 has been compressed into the reduced profile shape shown in FIGS. 8 and 9 and the side balloon 28 has been either folded or otherwise compressed in a desired manner upon the top side 34 of the main balloon 26, the balloons 26, 28 are prepared for assembly with other features of the catheter assembly 10. In such an assembly 10, the first and second non-compressible holding members 68, 70 are removed and the stent 16 is positioned over the main balloon 26 with the side balloon 28 positioned in axial and radial alignment with the side branch aperture 84 of the stent 16. The side catheter branch 14 is then advanced through the stent 16 and out of the side branch aperture 84. Later assembly steps include crimping the stent 16 upon the main and side catheter branches 12, 14 to help retain all of the catheter branch 12, 14 and stent 16 features together as a packaged catheter assembly 10 prepared for treatment of a vessel bifurcation in conjunction with use of the main and branch guidewires 18, 20.

### Materials and Other Considerations

The materials used in the balloons, catheter shafts, and edge protect members disclosed herein can be made of any suitable material including, for example, thermoplastic polymers, polyethylene (high density, low density, intermediate density, linear low density), various co-polymers and blends of polyethylene, ionomers, polyesters, polycarbonates, polyamides, poly-vinyl chloride, acrylonitrile-butadiene-styrene copolymers, polyether-polyester copolymers, and polyetherpolyamide copolymers. One suitable material is Surlyn®, a copolymer polyolefin material (DuPont de Nemours, Wilmington, Del.). Still further suitable materials include thermoplastic polymers and thermoset polymeric materials, poly(ethylene terephthalate) (commonly referred to as PET), thermoplastic polyamide, polyphenylene sulfides, polypropylene. Some other example materials include polyurethanes and block copolymers, such as polyamide-polyether block copolymers or amide-tetramethylene glycol copolymers. Additional examples include the PEBAX® (a polyamide/polyether/polyester block copolymer) family of polymers, e.g., PEBAX® 70D, 72D, 2533, 5533, 6333, 7033, or 7233 (available from Elf AtoChem, Philadelphia, Pa.). Other examples include nylons, such as aliphatic nylons, for example, Vestamid L21011F, Nylon 11 (Elf Atochem), Nylon 6 (Allied Signal), Nylon 6/10 (BASF), Nylon 6/12 (Ashley Polymers), or Nylon 12. Additional examples of nylons include aromatic nylons, such as Grivory (EMS) and Nylon MXD-6. Other nylons and/or combinations of nylons can also be used. Still further examples include polybutylene terephthalate (PBT), such as CELANEX® (available from Ticona, Summit, N.J.), polyester/ether block copolymers such as ARNITEL® (available from DSM, Erionspilla, Ind.), e.g., ARNITEL® EM740, aromatic amides such as Trogamid (PA6-3-T, Degussa), and thermoplastic elastomers such as HYTREL® (Dupont de Nemours, Wilmington, Del.). In some embodiments, the PEBAX®, HYTREL®, and ARNITEL® materials have a Shore D hardness of about 45D to about 82D. The balloon materials can be used pure or as blends. For example, a blend may include a PBT and one or more PBT thermoplastic elastomers, such as RITEFLEX® (available from Ticona), ARNITEL®, or HYTREL®, or polyethylene terephthalate (PET) and a thermoplastic elastomer, such as a PBT thermoplastic elastomer. Additional examples of balloon materials can be found in U.S. Pat. No. 6,146,356. It should be understood that the specific materials disclosed below for the individual embodiments does not limit the embodiment to those materials.

In the example catheter assemblies described above, some of the features can include a lubricious coating on an exterior surface thereof. The coating can promote insertion of the branch balloon into the branch vessel of a vessel bifurcation. The coating can also improve removal of the branch balloon from the branch vessel and the branch aperture of the stent when deflating and removing the catheter assembly from the vessel bifurcation after expansion of the stent. Some example coating for use with the branch balloon include hydrophilic polymers such as polyarylene oxides, polyvinylpyrolidones, polyvinylalcohols, hydroxyl alkyl cellulosics, algins, saccharides, caprolactones, and the like, and mixtures and combinations thereof. Hydrophilic polymers can be blended among themselves or with formulated amounts of water insoluble compounds (including some polymers) to yield coating with suitable lubricity, bonding and solubility. In some examples, portions of the devices described herein can be coated with a hydrophilic polymer or a fluoropolymer such as polytetrafluoroethylene (PTFE), better known as TEFLON®.

While the example stent delivery systems described above illustrate a balloon expandable stent having a predetermined side opening (i.e., branch aperture), other types of stents can be used with the catheter features described above. A variety of stents can be used with the systems and methods disclosed herein. Examples of such stents can be found in, for example, U.S. Pat. Nos. 6,210,429, 6,325,826, and 7,220,275, the entire contents of which are incorporated herein by reference. In general, the aforementioned stents have a tubular shape with a continuous sidewall that extends between the proximal and distal ends. Proximal and distal stent apertures are defined at respective proximal and distal ends of the stent. A branch aperture is defined in the sidewall of the stent. The branch aperture provides access between an interior of the stent and an exterior of the stent. In some stents, the branch aperture includes expandable structure around a peripheral edge thereof that expands in a generally radial outward direction relative to a longitudinal axis of the stent. The expandable structure can be configured to extend into the branch lumen of the bifurcation upon expansion of the stent. The stent includes a plurality of strut structures that define the sidewall. The struts are expandable from a first, unexpanded state to a second, expanded state. Typically, the stent is configured to maintain the expanded state. The struts define a plurality of cell openings or cells along a length of the stent. The size and shape of the cells is typically different than the size and shape of the branch aperture. The stent is typically expanded once the stent is properly positioned in the main lumen of the bifurcation with the branch aperture aligned radially and axially with an opening into the branch lumen. The stent, including the expandable structure surrounding the branch aperture, can be expanded with a single expansion or with multiple expansions using, for example, one or more inflatable balloons.

### CONCLUSION

One aspect of the present disclosure relates a catheter assembly that includes a stent and a first catheter branch. The stent has a distal open end, a proximal open end, and a side branch aperture. The first catheter branch includes a main balloon and a side balloon. The main balloon has opposing top and bottom portions, opposing first and second side portions, and opposing proximal and distal end portions. The side balloon is positioned on the top portion in alignment with the side branch aperture. The first and second side portions are folded in opposite directions towards the bottom portion.

Another aspect of the present disclosure relates to a catheter balloon assembly that includes a side balloon and a main balloon. The main balloon includes opposing top and bottom portions, opposing first and second side portions, and opposing proximal and distal end portions. The side balloon is positioned on the top portion of the main balloon and configured to extend radially outward relative to the main balloon when the side balloon is inflated. When the main balloon is in an uninflated state the first and second side portions are folded in opposite directions towards the bottom portion of the main balloon into a folded state.

A further aspect of the present disclosure relates to a method of folding a catheter balloon assembly. The catheter balloon assembly includes a main balloon and a side balloon, wherein the main balloon includes opposing proximal and distal end portions, opposing top and bottom portions, and opposing first and second side portions. The side balloon is positioned on the top portion and is configured to extend radially outward from the top portion of the main balloon when the side balloon is inflated. The method includes inflating the main balloon and the side balloon, applying a compressive force to the main balloon in a direction from the top portion towards the bottom portion, and folding the first and second side portions of the main balloon in opposite directions towards the bottom portion of the main balloon to place the main balloon in a first folded state.

The above specification, examples and data provide a complete description of the manufacture and use of the composition of the invention. Since many embodiments of the invention can be made without departing from the spirit and scope of the invention, the invention resides in the claims hereinafter appended.

We claim:

1. A catheter assembly, comprising:
a) a stent having a distal open end, a proximal open end, and a side branch aperture; and
b) a first catheter branch, the first catheter branch including a main balloon and a side balloon, the main balloon having opposing top and bottom portions, opposing first and second side portions, and opposing proximal and distal end portions, the side balloon being positioned on the top portion, wherein the first and second side portions are folded in opposite directions towards the bottom portion, and the side balloon is arranged in alignment with the side branch aperture.

2. The catheter assembly of claim 1, further comprising a second catheter branch extending through the proximal open end of the stent and out of the side branch aperture.

3. The catheter assembly of claim 1, wherein the first catheter branch further includes a main guidewire housing, the main guidewire housing defining a main guidewire lumen configured to house a main guidewire.

4. The catheter assembly of claim 2, wherein the second catheter branch defines a branch guidewire housing, the branch guidewire housing defining a branch guidewire lumen configured to house a branch guidewire.

5. The catheter assembly of claim 1, wherein the main balloon is compressed into a flattened configuration from the top surface to the bottom surface prior to the first and second side portions being folded into the folded state.

6. The catheter assembly of claim 1, wherein the side balloon is formed integral with the main balloon.

7. A catheter balloon assembly, comprising:
a) a side balloon; and
b) a main balloon, the main balloon having opposing top and bottom portions, opposing first and second side portions, and opposing proximal and distal end portions, the side balloon being positioned on the top portion of the main balloon and configured to extend radially outward relative to the main balloon when the side balloon is inflated, wherein when the main balloon is in an uninflated state the first and second side portions are folded in opposite directions towards the bottom portion of the main balloon into a folded state.

8. The catheter assembly of claim 7, wherein the folded state of the main balloon is maintained with a compressible holding member.

9. The catheter assembly of claim 7, wherein at least a portion of the side balloon is folded towards one of the proximal and distal end portions of the main balloon.

10. The catheter assembly of claim 7, wherein the side balloon is formed integral with the main balloon.

11. The catheter assembly of claim 7, wherein the folded state of the main balloon is maintained with a non-compressible holding member.

12. The catheter assembly of claim 7, wherein the main balloon is compressed into a flattened configuration wherein the top portion engages the bottom portion prior to the first and second side portions being folded into the folded state.

13. A method of folding a catheter balloon assembly, the catheter balloon assembly including a main balloon and a side balloon, the main balloon having opposing proximal and distal end portions, opposing top and bottom portions, and opposing first and second side portions, the side balloon being positioned on the top portion, the side balloon being configured to extend radially outward from the top portion of the main balloon when the side balloon is inflated, the method comprising:
a) inflating the main balloon and the side balloon;
b) applying a compressive force to the main balloon in a direction from the top portion towards the bottom portion; and
c) folding the first and second side portions of the main balloon in opposite directions towards the bottom portion of the main balloon to place the main balloon in a first folded state.

14. The method of claim 13, further comprising:
a) inflating the side balloon while maintaining the first folded state of the main balloon; and
b) applying a compressive force to the side balloon while deflating the side balloon to compress at least a portion of the side balloon toward the main balloon.

15. The method of claim 14, further comprising folding at least a portion of the side balloon after the step of applying the compressive force to the side balloon.

16. The method of claim 13, wherein maintaining the first folded state of the main balloon includes inserting at least a portion of the main balloon into a holding member.

17. The method of claim 13, further comprising applying a compression force to the main balloon after the folding step to reduce an outer profile of the main balloon.

18. The method of claim 17, further comprising inserting at least a portion of the main balloon into a compressible holding member prior to applying the compression force.

19. The method of claim 18, further comprising:
a) removing the compressible holding member from the main balloon after applying the compression force; and
b) inserting at least a portion of the main balloon into a non-compressible member after removing the main balloon from the compressible holding member.

20. The method of claim 13, further comprising deflating the main balloon during the step of applying a compressive force to the main balloon.
